(19) United States
(12) Patent Application Publication
(10) Pub. No.: US 2025/0090851 A1
(43) Pub. Date: Mar. 20, 2025

(54) Temporary External Pacemaker Visualization

(71) Applicant: Axiom Medical LLC, Springfield, OR (US)

(72) Inventor: Jacob Thomas McVicker, Springfield, OR (US)

(73) Assignee: Axiom Medical LLC, Springfield, OR (US)

(21) Appl. No.: 18/613,559

(22) Filed: Mar. 22, 2024

Related U.S. Application Data
(60) Provisional application No. 63/583,597, filed on Sep. 19, 2023.

(51) Int. Cl. A61N 1/362; A61N 1/05

(52) U.S. Cl. CPC A61N 1/3625; A61N 1/056; A61N 1/0595

(57) ABSTRACT

In some embodiments, a portable interface for a temporary external pacemaker comprising a housing, a communication module disposed in or on the housing for communicating with a pacemaker generator, and a display coupled to the housing, the display having a graphical interface to visualize a patient's heart rhythm and a sensitivity overlay that correlates with a sensitivity setting.

### RELATED APPLICATIONS

The present application claims priority to U.S. Ser. No. 63/583,597, filed Sep. 19, 2023, the disclosure of which is hereby incorporated by reference in its entirety as if fully set forth herein

### FIELD OF DISCLOSURE

The present disclosure relates to temporary external pacemakers. More particularly the present disclosure relates to devices and methods to aid in visualizing the settings of a temporary external pacemaker.

### BACKGROUND OF THE DISCLOSURE

Temporary external pacemakers are medical devices designed to manage and regulate a patient's heart rate when their natural cardiac electrical system is compromised or dysfunctional. These devices serve as a crucial lifeline in emergency situations or during the post-operative recovery phase when the heart's electrical conduction system is impaired. Temporary external pacemakers consist of a pulse generator, typically a small handheld device, and electrode leads that are attached to the patient's chest. The pulse generator emits electrical impulses that stimulate the heart muscle, initiating contractions and maintaining a regular heartbeat. These pacemakers are often used in critical care units, operating rooms, or during cardiac emergencies to stabilize patients with bradycardia (a slow heart rate) or other rhythm abnormalities.

Temporary external pacemakers are invaluable in scenarios where rapid intervention is required to support a patient's heart function. They must be properly set and adjusted to control the pacing rate and maintain optimal heart rate parameters, ensuring adequate blood flow and oxygen delivery to vital organs. While they are not a long-term solution, temporary external pacemakers buy time for healthcare providers to diagnose and address the underlying cardiac issues. Once the patient's condition stabilizes, more permanent solutions, such as implantable pacemakers, may be considered. Conventional temporary external pacemakers may be difficult to operate and adjusting the settings requires experience and care to ensure that the operation is safe for the patient.

### SUMMARY OF THE DISCLOSURE

In some embodiments, a portable interface for a temporary external pacemaker comprising a housing, a communication module disposed in or on the housing for communicating with a pacemaker generator, and a display coupled to the housing, the display having a graphical interface to visualize a patient's heart rhythm and a sensitivity overlay that correlates with a sensitivity setting.

### BRIEF DESCRIPTION OF THE DISCLOSURE

Various embodiments of the presently disclosed temporary external pacemakers are shown herein with reference to the drawings, wherein:

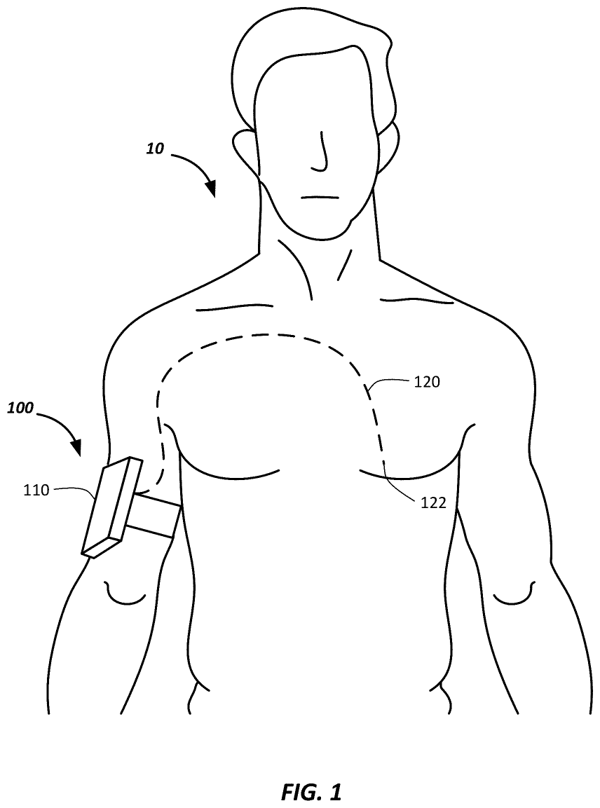

FIG. 1 is an anterior view of a patient that is being treated using a temporary pacemaker system in accordance with some embodiments provided herein;

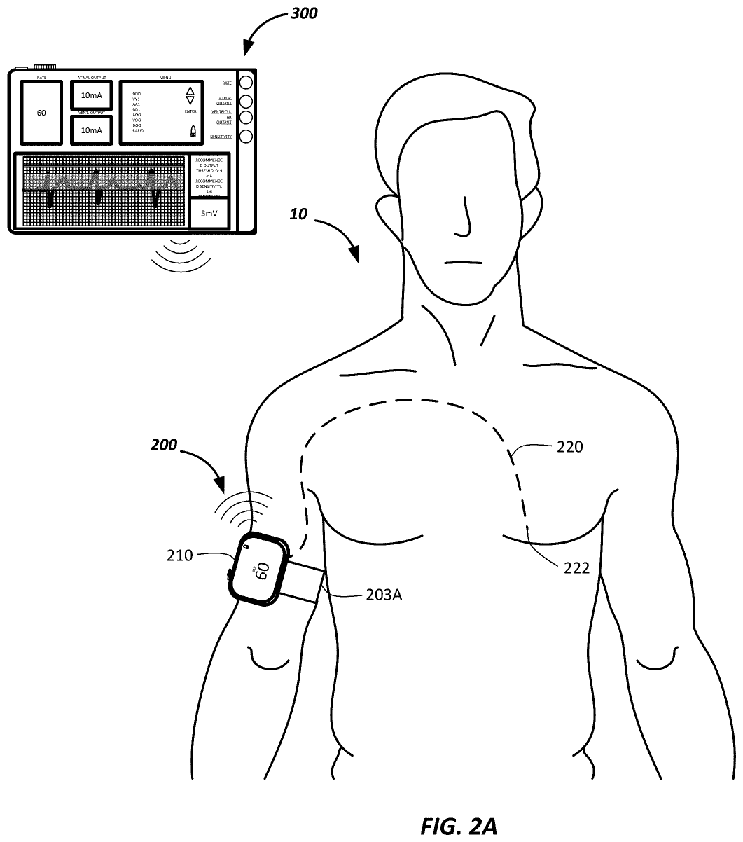

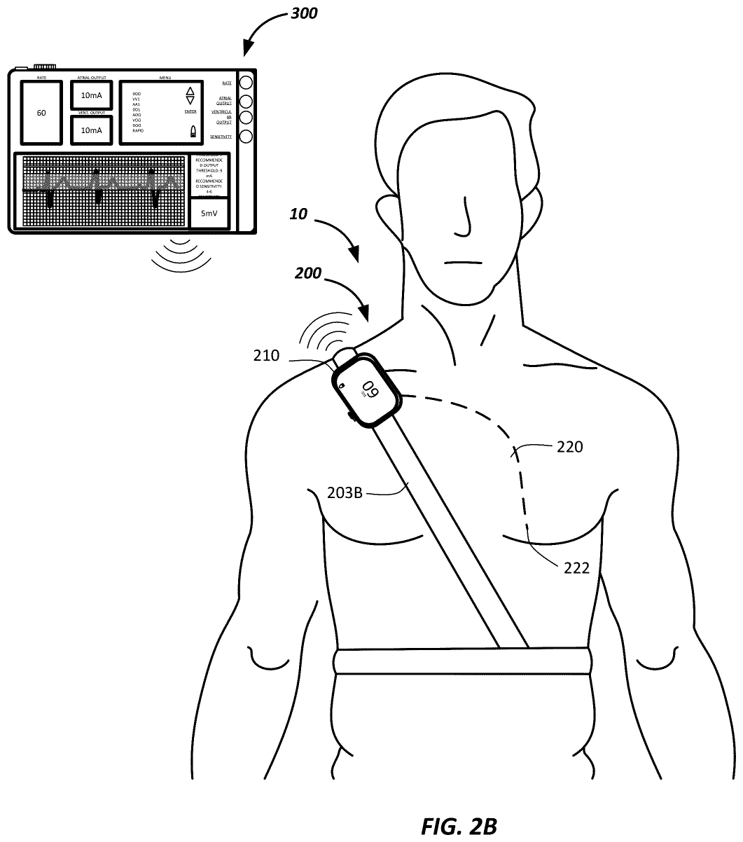

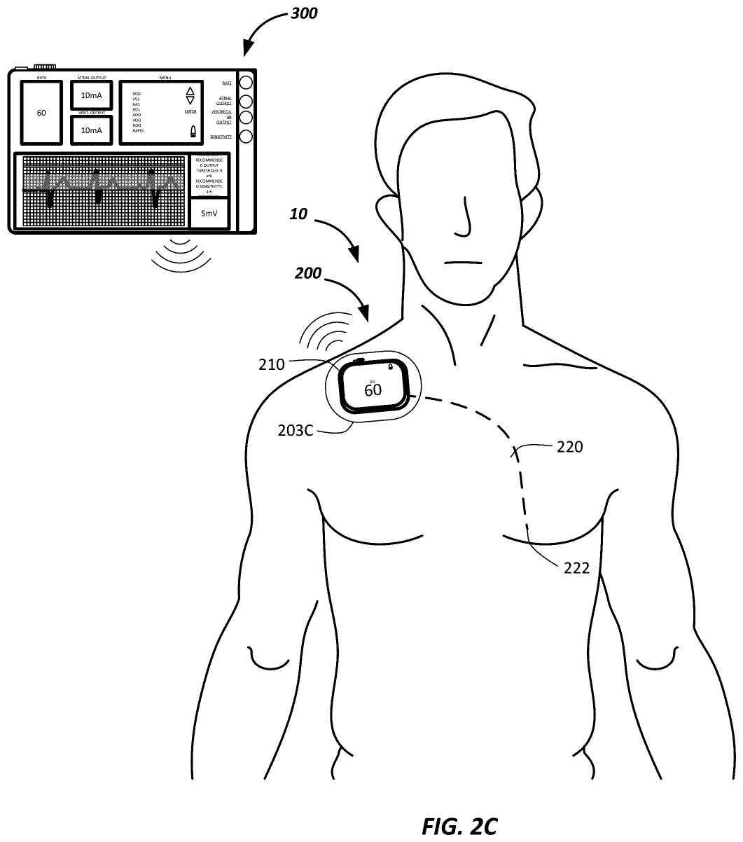

FIG. 2A-2C are anterior views of a patient that is being treated using a temporary pacemaker system in accordance with some embodiments provided herein;

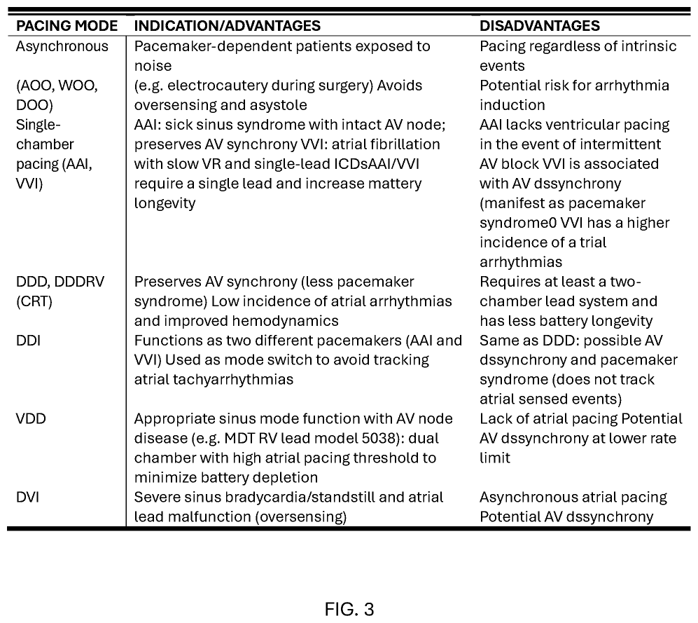

FIG. 3 is a table showing an external temporary pacemaker menu for selecting a pacing mode;

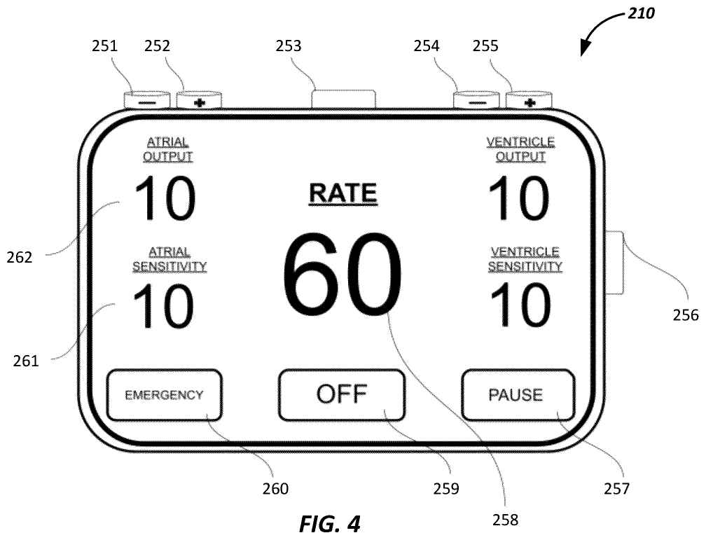

FIG. 4 is a schematic representation of a pacemaker pulse generator; and

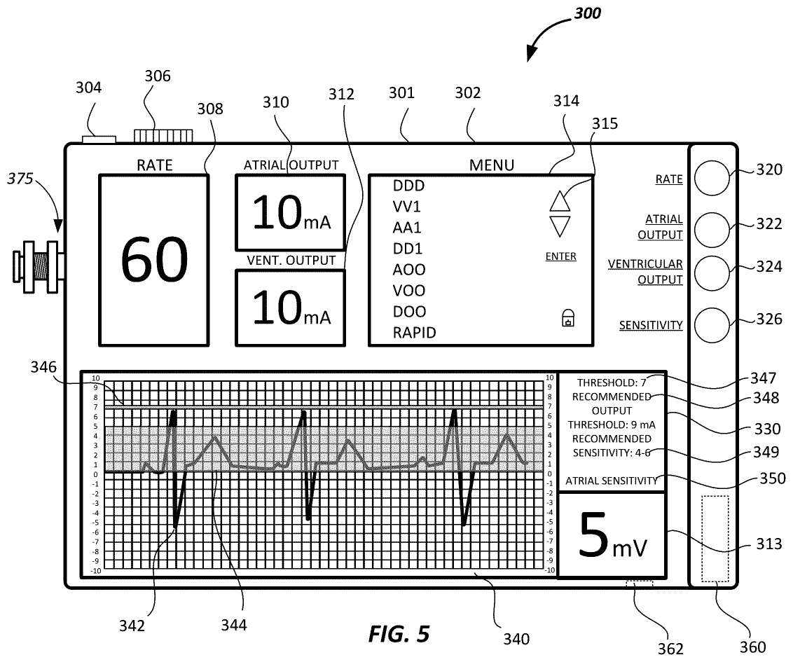

FIG. 5 is a schematic representation of a portable pacemaker interface to be used with a pulse generator.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to temporary external pacemakers and their methods of use, conventional devices suffer from some shortcomings.

There therefore is a need for further improvements to the devices, systems, and methods of using temporary external pacemakers. Among other advantages, the present disclosure may address one or more of these needs.

The present disclosure attempts to provide improvements in external temporary pacemakers by allowing for increased user compatibility and safety without compromising the continued pacemaker stimulation of the myocardium or heart muscle.

Referring to FIG. 1, a patient 10 is shown being treated by, for example, a transvenous temporary endocardial pacemaker system 100 in accordance with some embodiments. In the depicted embodiment, transvenous temporary endocardial pacemaker system 100 includes a pulse generator 110 and a transvenous pacing lead 120. Transvenous temporary endocardial pacemaker system 100 can provide pacing to the heart of patient 10 to treat arrhythmias. A tip 122 of transvenous pacing lead 120 can includes one or more electrodes (e.g., bipolar electrode pairs or one or more unipolar electrodes) that reside in contact with endocardial tissue (e.g., in the right ventricle). Transvenous pacing lead 120 can be designed for temporary use (e.g., for time periods of about one day to about three months).

The depicted arrangement utilizes venous access via a brachial artery of patient 10. In some cases, other venous access pathways can be used, such as, but not limited to, transjugular and transfemoral. The transbrachial access as depicted can provide mobility advantages in comparison to other venous access pathways.

Pulse generator 110 can be equipped for short-range wireless communications with monitoring equipment to enhance mobility of patient 10. Generator 110 can be re-chargeable and re-useable. In addition to electrodes, tip 122 of transvenous pacing lead 120 can include one or more anchoring and/or anti-migration features. Such anchoring and/or anti-migration features can be active or passive anchoring features, or a combination of both active and passive. For example, in some cases tip 122 can include one or more selectively deployable screws (e.g., spiral tines), hooks, barbs, or tines that are actively engageable with endocardial tissue. In some cases, tip 122 can include one or more tines that are passively engageable with endocardial tissue (e.g., like a grappling hook arrangement). In some cases, tip 122 is selectively expandable such that the electrodes and the anchoring and/or anti-migration features engage with tissue at the time of expansion of tip 122.

In some cases, the anchoring and/or anti-migration features (or portions thereof) of tip 122 are bioabsorbable (e.g., made of a dissolving polymer). The bioabsorbable aspect can facilitate retrieval of temporary transvenous pacing lead 120. In some cases, the anchoring and/or anti-migration features (or portions thereof) of tip 122 are electro-phasic polymer passive fixation tines that release when appropriate electrical energy is applied. Differing types of anchoring and/or anti-migration features can be used depending on the anticipated length of use (time period) of a particular temporary transvenous pacing lead 120.

In some implementations, tip 122 includes one or more features for visualizing or guiding tip 122 during the deployment process. For example, in some cases tip 122 includes one or more magnetic elements (e.g., electromagnets). Such magnetic elements can be used to facilitate guidance of temporary transvenous pacing lead 120 during transvenous deployment, as described further below. In some cases, tip 122 can include an ultrasound emitter/marker for use with ultrasound guidance during the deployment process.

In some implementations, tip 122 includes an impedance sensor, flow sensor, pressure sensor, or combinations thereof to provide an indication of when tip 122 is against an endocardial tissue surface. Moreover, in some implementations the electrodes of tip 122 can provide electrogram-based information that can be used for this purpose. In some cases, transvenous pacing lead 120 can be installed over a previously-placed guidewire. In some such cases, the guidewire can be installed using ultrasound (e.g., a hand-held device) for visualization.

As shown in FIG. 2A, in some cases, an external temporary pacemaker 200 may include a pulse generator 210 connected to a pacing lead 220 that houses electrodes at its tip 222. Pacing lead 220 may be positioned within the right atrium or right ventricle of the heart, or on the epicardium, as per the patient's requirements. Pulse generator 210 may produce pacing impulses through the distal electrodes. Additionally, the system may utilize the electrodes to monitor the electrical activity in the myocardium. Pulse generator 210 may be carried by the patient via a fixation element (e.g., an arm strap 203A as shown in FIG. 2A, a chest strap 203B as shown in FIG. 2B and/or an adhesive patch 203C as shown in FIG. 2C). A portable interface unit 300 may be in communication (e.g., via a wired connection, Bluetooth or other communication protocol) with pulse generator 210 and may be configured to adjust the settings on the generator 210.

Generally, external temporary pacemakers feature settings such as Output (mA), Rate (BMP, beats per minute), and Sensitivity (mV). The output, in mA, is the required current of electricity needed to capture or produce a heartbeat. The rate or BPM, which reflects the frequency of the beats, is ordered by the provider and set by the medical staff. The sensitivity, in mV, is set so that the pacemaker can sense the electrical activity at the myocardial surface. The sensitivity may be defined as the minimum myocardial voltage required to be detected as a P wave (atrial wave) or R wave (ventricular wave). The sensitivity setting is of utmost importance for allowing the pacemaker to sense a patient's intrinsic, natural heartbeat, and for determining whether a paced beat should be provided. Typically, the sensitivity is set by the nursing staff during the placement and continued care of the patient while an external temporary pacemaker is in place. All of these settings may be adjusted via the interface 300. Additionally, the pulse generator 210 and/or interface 300 may include a menu for selecting provider-ordered pacing modes such as atrial burst pacing or other modes as shown in FIG. 3, and as will be described in greater detail below.

Turning to FIG. 4, one example of a pulse generator 210 is shown according to an embodiment of the disclosure. Generator 210 may include a negative atrial lead connector port 251 and a positive atrial lead connector port 252, as well as a negative ventricle lead connector port 254 and a positive ventricle lead connector port 255. Generator 210 may also include a Bluetooth connectivity device 253 (or other communication protocol for) for wirelessly communicating with interface unit 300. A wired connection port 256 may also be capable of connecting to interface unit 300. Generator 210 may also include a pause button 257, a rate display 258 that shows the set pacemaker rate control, and an off button 260. In some examples, pulse generator 210 includes an emergency button 260 for asynchronous pacing. Pulse generator 210 may also include two displays showing lead sensitivity settings including a ventricular output sensitivity setting 261 and an atrial output sensitivity setting 262.

Sensitivity, pertaining to a pacemaker electrode, refers to the minimal myocardial voltage necessary for detection as a P wave or R wave. This sensitivity is quantified in millivolts (mV). Skilled nursing staff have control over the manual sensitivity setting of a temporary pacemaker. It can be adjusted to a high setting, resulting in undersensing, where the pacemaker fails to recognize the P wave or R wave of the patient's intrinsic heartbeat. Conversely, it can be set to a low sensitivity, leading to oversensing, where the pacemaker registers other electrical activity as the patient's intrinsic heartbeat and refrains from pacing. The instant disclosure may enhance patient safety and streamline usability for skilled nursing staff. This is achieved by employing calculated ranges and visual aids to configure pacemaker settings to their most optimal levels.

As shown in FIG. 5, in some embodiments, a portable programmer interface 300 may include features or options to quickly adjust settings and visualize the functionality of pulse generator 210. While pulse generator 210 may have certain controls, the vast majority of pulse generator settings may be adjusted from the interface. Interface 300 may include a housing 301, a display 302 having a Bluetooth connection module 304, and/or a plug 306 for connecting to a generator 210. Plug 306 may include wired connection ports originating from temporary pacemaker catheters that allow interface 300 to function as the primary pacemaker, taking precedence over pulse generator 210 in case of an emergency.

In this example, interface 300 may include a plurality of boxes or modules such as a large text readout 308 of the rate, and a large text readout of the atrial output 310 and the ventricular output 312. A sensitivity readout box 313 is also shown in the lower right-hand corner. The interface may also include a menu 314 having buttons, arrows or toggles 315 for selecting provide-ordered pacing modes such as atrial burst pacing or other modes previously shown in FIG. 3. Buttons or knobs 320-326 may be used to adjust the rate 320, atrial output 322, ventricular output 324 and/or sensitivity 326, for example, by turning them clockwise or counter-clockwise or toggling them.

In accordance with some embodiments, the function of the pacemaker programmer interface 300 includes improved visuals on the screen to promote patient safety and increased user compatibility. In FIG. 5, interface 300 further includes a visual graph feature 340 within display screen 302 that features an electrocardiogram graph of the patient's heart rhythm. Specifically, graph feature 340 may visualize the sensed current heart rhythm 342 displayed in real time when the programmer interface is turned on. The heart rhythm 342 may be obtained from an atrial signal if the catheter lead is placed in the atrium, or a ventricular rhythm if the lead is placed in the ventricle. Specifically, a tip of transvenous pacing lead may include one or more electrodes (e.g., bipolar electrode pairs or one or more unipolar electrodes) that reside in contact with endocardial tissue (e.g., in the right ventricle). Electrodes in contact with endocardial tissue send a signal to pulse generator 210 and transmit said signal to interface 300 which converts said signal into an ECG on graph feature 340. During insertion and continued use of the temporary pacemaker, the interface 300 would provide an ECG on graph feature 340 consistent with current placement of lead, improving physician and skilled nursing interpretation of lead current location (e.g., superior vena cava, right atrium, right ventricle), Quickly identifying lead's current location through the ECG on graph feature 340 may confirm correct placement and/or displacement without the need for other medical instruments. The graph feature 340 is featured on standard ECG paper scale with 1 mV represented by a deflection of 10 mm. Thus, each 1 mm square on the vertical axis equals 0.1 mV. The graph feature 340 may include a shaded or transparent overlay 344 that correlates with the current sensitivity setting. When the user adjusts the sensitivity of the pacemaker using knob 326 for example, the sensitivity box 313 will show the new set sensitivity value. Concurrently, actuating knob 326 will also increase or decrease the location of transparent overlay 344 in increments of 1 mm box or 0.1 mV as the sensitivity is set. The transparent overlay 344 feature may provide an intuitive visual representation of the sensitivity setting and myocardial capture within the same interface for improved nurse use and improved patient safety. In some examples, ECG sensitivity transparent overlay 344 offers a visual representation in millivolts (mV) of the current sensitivity setting. In transparent overlay 344, the region below the current sensitivity setting down to 0 mV will be shaded so as to be identifiable but at least partially transparent so that the heart rhythm 342 can be seen therethrough (e.g., a region with 25%, 50% or 75% opacity), aiding in the visualization of ECG waves that the pacemaker can detect (above the line) and those it cannot (below the line and in the transparent field). This grants skilled nursing staff a clear depiction of sensitivity settings, ensuring the pacemaker neither oversenses nor undersenses intrinsic rhythm, and adjustments can be made based on the transparent overlay.

The pacemaker programmer interface 300 may also feature a display screen of calculated recommended settings 330 for medical staff interpretation. The calculated recommended settings 330 may include a calculated range so that medical staff can better determine if they are within the pacemaker's range of output and sensitivity settings. These recommended ranges provide a secondary check to the graph overlay for medical providers. In some examples, recommended settings 330 may include a recommended output threshold 348, which is an averaged range calculated based on ongoing output measurements taken at regular intervals over a specific period of time. This recommended output may serve as a crucial safety guideline for skilled nursing staff, assisting them in accurately configuring the pacemaker output for the designated pacing area, be it the atrium or ventricle. Adhering to the recommended range, confirmed on the corresponding ECG graph, can boost nurse confidence and save time.

Recommended settings 330 may also include a device-calculated threshold 347, a numeric value derived from the average threshold computed during routine intervals over a defined period. It may also include a recommended sensitivity range 349 (e.g., between 4-6), which may be an averaged range derived from ongoing sensitivity measurements taken by the device at regular intervals over a specific duration. This recommended sensitivity range acts as an essential safety measure for skilled nursing staff during sensitivity adjustment. A setting within this recommended range, verified on the corresponding ECG graph via overlay 344, enhances nurse confidence and efficiency. If a setting falls outside this range, it would prompt skilled nursing staff to verify on the ECG and either adjust the setting or seek expert advice. Below recommended sensitivity range 349 is a notation 350 that designates the current portion of the heart displayed in the ECG graph, which can be switched to either the atrial lead or ventricular lead based on user preference. When a lead is selected, the sensitivity setting will automatically populate in sensitivity box 313.

Additionally, a visualized threshold value 346 may also be shown. In some examples, the threshold value 346 is a direct line representation of device-calculated threshold 347. The direct line representation of threshold 347 by line 346 improves safety by providing nurses a secondary projection of numerical text threshold 347 on the ECG graph 340 to confirm output setting 310, 312 are set higher to obtain appropriate myocardial capture.

As briefly noted, interface 300 may include Bluetooth connection 304. In some examples, generators 210 of the present disclosure may benefit from a wired or wireless configuration to the pacemaker programmer interface 300, with a Bluetooth-adapted pacemaker interface that features a screen and visuals of current pacemaker settings for easy viewing. The generator 210 may be worn in a sling, brace or strap (e.g., arm strap, adhesive patch, chest strap, etc.) to allow for patient mobility for improved outcomes as bedridden patients may suffer from immobility, muscle mass reduction, physical impairment, bone mineral density problems, and/or a longer stay. A wired connection port may allow for quick and easy pairing of the interface 300 to a pulse generator 210 case of emergency. The pacemaker programmer interface 300 may also feature an internal battery 360 and/or an external charging port 362. Similarly, pacemaker generator may feature an internal battery, external battery, and/or an external charging port. The programmer interface may also feature one or more clamp 375 disposed on one side of the device for easy IV pole placement and vertical rotation of interface for improved ergonomics for nursing staff.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A portable interface for a temporary external pacemaker comprising:
a housing;
a communication module disposed in or on the housing for communicating with a pacemaker generator; and
a display coupled to the housing, the display having a graphical interface to visualize a patient's heart rhythm and a sensitivity overlay that correlates with a sensitivity setting.

2. The portable interface of claim 1, wherein the communication module comprises Bluetooth.

3. The portable interface of claim 1, wherein the sensitivity overlay comprises a partially transparent overlay that offers a visual representation in millivolts of the current sensitivity setting.

4. The portable interface of claim 3, wherein the sensitivity overlay comprises an upper bound, a lower bound, and a shaded region between the upper bound and the lower bound.

5. The portable interface of claim 4, wherein the lower bound is 0 mV.

6. The portable interface of claim 4, wherein the upper bound represents a user-selected sensitivity setting.

7. The portable interface of claim 1, further comprising a device-calculated threshold derived from an average threshold computed during routine intervals over a defined period.

8. The portable interface of claim 1, further comprising a recommended output comprising an averaged range calculated based on ongoing output measurements taken at regular intervals over a specific period of time.

9. The portable interface of claim 1, further comprising a recommended sensitivity range comprising an averaged range calculated from ongoing sensitivity measurements taken by the device at regular intervals over a specific duration.

10. The portable interface of claim 1, further comprising a wired connection port for communicating with the pacemaker generator.

11. The portable interface of claim 1, further comprising a clamp coupled to the housing and mountable on a pole.

12. A system comprising:
the portable interface of claim 1; and
a portable generator.

13. The system of claim 12, wherein the portable generator comprises a negative atrial lead connector port, a positive atrial lead connector port, a negative ventricle lead connector port and a positive ventricle lead connector port.

14. The system of claim 12, wherein the portable generator comprises a Bluetooth module for communicating with the portable interface.

15. The system of claim 12, wherein the portable generator comprises two displays reflecting a ventricular output sensitivity setting and an atrial output sensitivity setting.

16. The system of claim 12, wherein the portable generator comprises a display showing a set pacemaker rate control.
